(19) United States
(12) Patent Application Publication
(10) Pub. No.: US 2024/0093284 A1
(43) Pub. Date: Mar. 21, 2024

(54) QUANTIFICATION OF CELLS EMBEDDED IN A 3D SCAFFOLD

(71) Applicant: Avant Meats Company Limited, Hong Kong (CN)

(72) Inventors: Po San Mario Chin, Hong Kong (CN); Kai Yi Carrie Chan, Hong Kong (CN); Chun Hei Poon, Hong Kong (CN)

(73) Assignee: Avant Meats Company Limited, Hong Kong (CN)

(21) Appl. No.: 18/010,143

(22) PCT Filed: Sep. 11, 2021
(86) PCT No.: PCT/IB2021/058274

§ 371 Date: Dec. 13, 2022

Related U.S. Application Data
(60) Provisional application No. 63/080,662, filed on Sep. 18, 2020.

(51) Int. Cl. C12Q 1/686; C12Q 1/6806

(52) U.S. Cl. CPC C12Q 1/686; C12Q 1/6806

(57) ABSTRACT

A method for quantifying cells embedded in a scaffold, comprising the steps of (a) a lysing step to cause the cells to lyse using alkali or proteinase; (b) a purification step comprising centrifugation or a series of elution; and (c) a quantification step comprising the steps of (i) simultaneously carrying out probe-based qPCR on a sample reaction mix and a plurality of reference reaction mixes; (ii) collecting the signals from the probe-based qPCR products from the sample reaction mix and reference reaction mix; (iii) creating a standard curve based on the collected signals from the reference reaction mixes; and (iv) deducing the number of cells in the sample through the standard curve.

### CROSS-REFERENCE TO RELATED APPLICATION

This application claims priority to a U.S. Provisional Application Ser. No. 63/080,662, filed on Sep. 18, 2020, which is incorporated by reference herein in its entirety.

### TECHNICAL FIELD

Embodiments discussed herein generally relate to an assay method. In particular, the invention relates to the quantification of cells. Specifically, the invention relates to the quantification of cells in a 3D scaffold.

### BACKGROUND

For decades, 2D cell culture has been the major tool for cell research and tissue engineering due to its simplicity and easy measurement. However, it fails to provide a precise model of the in vivo environment of an animal or human body. In contrast, 3D scaffold cell culture mimics the in vivo environment. It not only provides mechanical support to cells but also mimics the extracellular matrix (ECM) to promote cell growth and differentiation.

Despite the benefits of 3D scaffold cell culture, there are several drawbacks compared to 2D cell culture. One of them is that quantification of cell growth within a scaffold has been challenging. One of the current approaches involves labeling the cells with fluorescence probes embedded in a scaffold, imaging a small area of the scaffold (˜1 mm) under a microscope, followed by counting the number of cells in the image either manually or by the image analysis software.

However, there are some downsides to this approach. First, not every type of scaffolding material may be imaged. Cells embedded in an opaque or translucent scaffold cannot be imaged and counted. Further, cells are often labeled by fluorescence-tagged antibodies before imaging. This needs to be carefully optimized; samples need to be preserved in fixatives; antibodies must penetrate the sample and bind to specific epitopes on cells. Some cells may not be labeled if they are buried deep within the sample or if the epitopes are destroyed during sample processing. Performance or specificity of antibody varies leading to false labeling. Further, given that imaging methods only image several areas of the scaffold to obtain an average cell count, an uneven cell distribution will likely cause data inaccuracy.

In addition, imaging a large sample or multiple samples is time-consuming. Cell quantification by imaging is inefficient if the sample size is large (i.e. >100). Also, post-imaging analysis is time-consuming and labor-intensive. If cells are counted manually, this introduces human error. Automatic cell counting by image analysis software also requires trial and error with the analysis parameters (e.g. defining object size, circularity, background noise). Different batches of samples require different analysis settings and thus cannot be consistently compared.

### SUMMARY

In the light of the foregoing, aspects of the present invention provide an alternative method to improve data accuracy and consistency when quantifying cell growth in 3D scaffold cell culture. In some embodiment, a reduction of overall time and manpower required for cell quantification in 3D scaffold cell culture is achieved.

Aspects of the invention overcome the limitations of the imaging methods as it does not require imaging with the microscope and thus translucent or opaque scaffolding material will not interfere with the detection. Also, uneven cell distribution in the scaffold will not cause a significant error as in the traditional imaging approach because the present invention covers substantially every cell in the entire scaffold.

According to some embodiments of the present disclosure, A method for quantifying cells embedded in a scaffold, comprising (a) a lysing step to cause the cells to lyse, wherein the lysing step comprises the step of heating the cells together with the scaffold in the present of a proteinase with a buffer or alkali; (b) a purification step comprising performing at least three times of elution and collecting all the eluates or performing centrifugation, thereby capturing at least 90% of genomic DNA released from the cells; and (c) a quantification step comprising the steps of (i) forming a sample reaction mix by adding a predetermined volume of the collected eluate or supernatant into a qPCR master mix; (ii) forming a plurality of reference reaction mixes wherein each of the reference reaction mix contains genomic DNA purified from a known number of cells diluted to different concentration; (iii) carrying out probe-based qPCR on the sample reaction mix and the plurality of reference reaction mixes; (iv) collecting the signals from the probe-based qPCR products from the sample reaction mix and reference reaction mixes; (v) creating a standard curve based on the collected signals from the reference reaction mix wherein the standard curve provides a correlation between the number of cells and the collected signals; and (vi) deducing the number of cells in the sample through the standard curve.

According to another embodiment of the present disclosure, the quantification step further comprises carrying out qPCR in a multiwell plate.

### BRIEF DESCRIPTION OF THE DRAWINGS

The disclosure may be better understood by reference to the detailed description when considered in connection with the accompanying drawings. The components in the figures are not necessarily to scale, emphasis instead being placed upon illustrating the principles of the disclosure.

FIG. 1 is a flowchart of a method for cell quantification of a 3D scaffold according to some embodiments of the present invention.

FIG. 2 shows amplification curves in a probe-based qPCR assay of five-fold serial dilutions of HEK-293 cells and a standard curve thereof according to one embodiment of the present invention.

FIG. 3 shows amplification curves in a probe-based qPCR assay of serial dilutions of HEK-293 cells embedded in sodium alginate scaffold using Spin Column Extraction and a standard curve thereof according to one embodiment of the present invention.

FIG. 4 shows amplification curves in a probe-based qPCR assay of serial dilutions of HEK-293 cells embedded in sodium alginate scaffold using Alkaline Extraction and a standard curve thereof according to one embodiment of the present invention.

FIG. 5 shows amplification curves in a probe-based qPCR assay of serial dilutions of HEK-293 cells seeded into denatured collagen scaffold using Spin Column Extraction and a standard curve thereof according to one embodiment of the present invention.

FIG. 6 shows an amplification curve in a probe-based qPCR assay of serial dilutions of HEK-293 cells seeded into denatured collagen scaffold using Alkaline Extraction and a standard curve thereof according to one embodiment of the present invention.

FIG. 7 shows a graph showing cell counts for cells growth within denatured collagen scaffold obtained from manual cell count, probe-based qPCR assay (using Spin Column Extraction), and probe-based qPCR assay (using Alkaline Extraction) on days 1, 5 and 8 respectively.

### DETAILED DESCRIPTION

Provided are materials and methods for cell quantification of a 3D scaffold. The scaffold may be 3D solid/semi-solid material. Methods of the invention may be used for quantifying any cells, including muscle cells, somatic cells, stem cells. The cells may be from any organism. Normal and/or tumor cell cultures may be quantified using the methods of the present invention. Similarly, the present invention may be used to quantify cell growth in cultivated meat or tissue transplants.

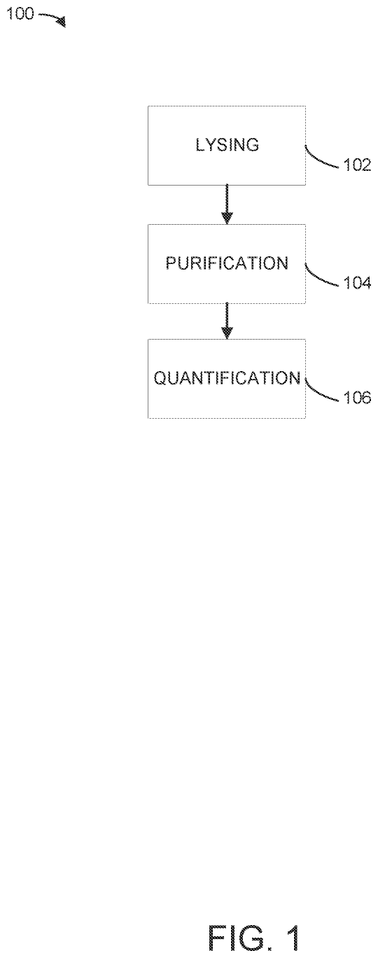

Referring to FIG. 1, methods of the present invention 100 generally involve lysing step 102, purification step 104 and quantification step 106. In the lysing step 102, the cells with the 3D scaffold are heated in the presence of an alkali or a proteinase, causing the cells in the cell cultures to lyse. The cells with the 3D scaffold could contain an unknown number of cells (Sample) and/or a known number of cells (Reference). In purification step 104, the genomic DNA (gDNA) released in the lysing step 102 are purified through centrifugation or a series of elution. In the quantification step 106, the purified gDNA from the Sample and the Reference in the purification step 104 are quantified using probe-based quantitative real-time PCR (Probe-based qPCR), where the amount of qPCR product from the Sample is compared to the amount of Probe-based qPCR product from a serial dilution of gDNA of the Reference in the same or separate Probe-based qPCR run. The purified gDNA obtained from the Reference may undergo a serial dilution prior to the Probe-based qPCR and all of the dilutions may undergo Probe-based qPCR.

A standard curve for the Reference (Reference Standard Curve) is generated based on the threshold cycles (Crossing Points) of the amplification curves of the dilutions of the Probe-based qPCR. The Reference Standard Curve provides information on the relationship between the quantity of cells in the dilutions and the Crossing Points. The quantity of cells in the Sample may be deduced from the Reference Standard Curve based on its Crossing Point in the amplification curve.

In some embodiments, the scaffold may be gel slice, sodium alginate, denatured collagen, calcium chloride or any other commercially available scaffold.

In some embodiments, the cells are derived from bony fish of the class Osteichthyes including saltwater fish such as a grouper, sea bass, or a yellow cocker. In yet other embodiments, the cells are derived from other types of animal tissue, such as cow tissue. In some embodiments, the cells may be derived from organ tissue, such as a swim bladder, from a fish.

Lysing

Lysing may be carried out by proteinase or alkali. Lysing the cells using proteinase is now described.

Lysing Cells Using Proteinase

In the lysing step 102, according to one embodiment of the present invention, the cells together with the scaffold are heated in the presence of proteinase with buffer, causing the cells in the cell culture to melt and lyse. The cells with the scaffold may be taken from the Sample. It is heated at about 55° C. for 1-16 hours with occasional (about every 15-60 minutes) vortexing, wherein each vortexing is less than 10 seconds. In some embodiments, it is heated at about 55° C. for about 8 hours with vortexing every about one hour, wherein each vortexing is about 5 seconds.

After the heating, the solution is centrifuged at 12000×g to 15000×g for about 2-5 minutes, preferably, at about 14000×g for about 3 minutes. The supernatant is added into RNase A, which is then incubated at room temperature (about 20° C.-26° C., preferably at about 24° C.) for about 2-5 minutes, preferably, 2 minutes. In some embodiments, the supernatant is added into 0.01 μl-5 ml RNase A. In a particular embodiment, the supernatant is added into 20 μl RNase A. Subsequently, lysis/binding buffer is added into the mixture above and vortexed for about 5 seconds. In some embodiments, about 0.01 μl-5 ml of lysis/binding buffer is added into the mixture. In a particular embodiment, 200 μl of lysis/binding buffer is added into the mixture above. Further, absolute ethanol is added to the solution and vortexed for about 5 seconds. In some embodiments, 0.1 μl-5 ml of absolute ethanol is added to the solution. In a particular embodiment, about 200 μl of absolute ethanol is added to the solution.

Any protease may be used in the lysing step described herein. In some embodiments, proteases of a thermophilic bacterium such as Thermus Aquaticus, Thermus fififormis, Thermotoga neapolitana, Thermotoga maritime and Thermococcus zilligi, (e.g., Thermus sp. strain RT41a thermostable alkaline protease and EA1 protease is used. In some embodiments, Proteinase K is used.

The buffer with proteinase may include PureLink™ Genomic Digestion Buffer. In some embodiments, the buffer used together with proteinase may contain a denaturing agent (e.g. guanidine thiocyanate or guanidine thiocyanate) and a detergent (e.g. Tween-20, Triton-X100, SDS or NP-40).

In some embodiments, the lysis/binding buffer may include PureLink™ Genomic lysis/binding buffer. In yet another embodiment, the lysis/binding buffer may contain denaturing agent (e.g. guanidine thiocyanate or guanidine thiocyanate).

Alternatively, lysing may be carried out by using alkali. Lysing the cells by using alkali is now described.

Lysing the Cells with Alkali

The lysing step 102, according to another embodiment of the present invention, the weight of the scaffold (with the cells adhered thereon) may be first obtained. Thereafter, the scaffold (with the cells adhered thereon) is frozen and stored at temperature in the range of −15° C. to −50° C. for 1-72 hours, preferably, at −20° C. for 1 hour. The scaffold (with the cells adhered thereon) is then thawed at room temperature. It may be minced during or after the thawing. Thereafter, the thawed and/or minced scaffold (with the cells adhered thereon) may be submerged under an alkali (for example, sodium hydroxide, potassium hydroxide, calcium hydroxide, Ammonia) at a concentration ranging from 40 nM to 80 nM, preferably 50 nM of Sodium hydroxide (NaOH). The mixture is then heated in a range of 80° C. to 100° C. for 15-120 minutes, preferably, at 98° C. for 30 minutes. The mixture may be occasionally vortexed for less than 10 seconds during the heating process. After heating, MilliQ water is added to the mixture. For some embodiments, 0.01 μl-5 ml MilliQ water is added to the mixture. In a particular embodiment, 600 μl of MilliQ water is added to the mixture. The mixture may be vortexed for less than 10 seconds after MilliQ water is added. Thereafter, Tris-HCL is added to the mixture. The concentration of the Tris-HCL maybe 0.5-3 M and pH maybe 7.0-9.0, preferably, 1 M and the pH may be at 8.0. The volume of the Tris-HCL added to the mixture maybe around 8%-15% of the total volume of MilliQ water added in the MilliQ water adding step, preferably 10%. The mixture may be vortexed for less than 10 seconds after adding Tris-HCL.

Purification

The lysing step 102 causes the cells to lyse and causes the contents of the cells, including the gDNA, to be released. Purification step 104 helps to purify the gDNA for the quantification step 106. The purification step 104 is different based on the way the cells were lysed.

The following purification step 104 is used if the cells were lysed by using proteinase (together with the proteinase lysing step, Spin Column Extraction).

The lysate from the proteinase lysing step is transferred to the spin column and centrifuge from 8000×g to 12000×g for 0.5-3 minutes, preferably, at 10000×g for about 1 minute. A first ethanol containing wash buffer is added to the spin column and centrifuge from 8000×g to 12000×g for 0.5-3 minutes, preferably, at 10000×g for about 1 minute. In some embodiments, 0.01 μl-5 ml of a first ethanol containing wash buffer is added to the spin column. In particular embodiment, 500 μl of a first ethanol containing wash buffer is added to the spin column. Subsequently, a second ethanol containing wash buffer is added to the spin column and centrifuged from 8000×g to 12000×g for 0.5-3 minutes, preferably, at 10000×g for about 1 minute. In some embodiments, 0.01 μl-5 ml of a second ethanol containing wash buffer is added to the spin column. In particular embodiment, 500 μl of a second ethanol containing wash buffer is added to the spin column. Upon completion, the spin column is further centrifuged from 8000×g to 12000×g for 1-5 minutes, preferably, at 10000×g for about 3 minutes to remove substantially all wash buffer residual.

The gDNA released are then eluted by adding autoclaved Milli-Q water to the spin column and incubated at room temperature (about 20° C.-26° C., preferably at about 24° C.) for 1 minute. In some embodiments, the gDNA released are eluted by adding 0.1 μl-5 ml autoclaved Milli-Q water to the spin column. In particular embodiment, the gDNA released are eluted by adding 200 μl autoclaved Milli-Q water to the spin column. Subsequently, the mixture is centrifuged from 10000×g to 16000×g for 0.5-3 minutes, preferably, at 14000× g for 2 minutes. The first eluate is collected. In some embodiments, the first eluate typically contains about 55%-70% of the total gDNA released in the cells in the Sample.

A second elution is performed by adding autoclaved Milli-Q water to the spin column. In some embodiments, the second elution is performed by adding 0.1 μl-5 ml autoclaved Milli-Q water to the spin column. In particular embodiment, the second elution is performed by adding 200 μl autoclaved Milli-Q water to the spin column. Subsequently, the mixture is centrifuged from 10000×g to 16000×g for 0.5-3 minutes, preferably, at 14000×g for 2 minutes. The second eluate is collected. In some embodiment, the second eluate typically contains about 15%-25% of the total gDNA released in the cells in the Sample.

A third elution is performed by adding autoclaved Milli-Q water to the spin column. In some embodiments, the third elution is performed by adding 0.1 μl-5 ml autoclaved Milli-Q water to the spin column. In particular embodiment, the third elution is performed by adding 200 μl autoclaved Milli-Q water to the spin column. Subsequently, the mixture is centrifuged from 10000× g to 16000×g for 0.5-3 minutes, preferably, at 14000×g for 2 minutes. The third eluate is collected. In some embodiment, the third eluate typically contains about 5%-15% of the total gDNA released in the cells in the Sample.

The first, second and third eluates are then combined to form a final eluate. In some embodiments, the combined eluate typically contains about 90% of the total gDNA released in the cells in the Sample or Reference. In yet some embodiments, additional elution may be performed. The additional elution may be run in the same condition as the third elution.

In some embodiments, the first ethanol-containing wash buffer comprises sodium acetate in about 75% ethanol solution. In another embodiment, the second ethanol containing wash buffer comprises sodium acetate in about 90% ethanol solution.

The following purification step 104 is performed if the cells were lysed by using alkali (together with the alkali lysing step, Alkaline Extraction).

The lysate from the alkalis lysing step is centrifuge from 10000×g to 16000×g for 2-7 minutes, preferably, at 14000×g for about 5 minutes. The supernatant is then extracted. The supernatant containing the gDNA is further diluted with autoclaved MilliQ water around 9-12 times before loading it to qPCR plate in the quantification step, preferably 10 times. gDNA in the supernatant could be from the cells in the Sample or Reference. The final supernatant typically contains about 90% of the total gDNA released in the cells in the Sample or Reference. In some embodiments, 0.1 μl-5 ml autoclaved MilliQ water is added into the supernatant per 0.1 μl-5 ml supernatant. In one particular embodiment, 900 μl autoclaved MilliQ water is added into the supernatant per 100 μl supernatant.

Quantification

The term “primer” refers to a single-stranded oligonucleotide sequence complementary to the nucleic acid strand to be copied and capable of acting as a point of initiation for the synthesis of a primer extension product.

The term “probe” refers to a single-stranded oligonucleotide sequence complementary to the qPCR product and contains a 5′ fluorophore and a 3′ quencher. The probe may contain an internal quencher.

qPCR is carried out to quantify GAPDH gDNA in the cell culture. Other genes can be used for quantitation e.g. EF1A1. They are selected as the target gene gDNA to produce a reliable result in the quantification of cells. The primers and probe used to quantify GAPDH are shown in Table 1.

TABLE 1

| Target gene | | Amplicon |
|---|---|---|
| gDNA | Primer/qPCR Probe sequence | size |
| Human Gapdh | F: 5′-TCAAGAAGGTGGTGAAGCAG | 117 bp |
| primers | G-3′ | |
| | R: 5′-CGTCAAAGGTGGAGGAGTG | |
| | G-3′ | |
| Human Gapdh | 5′-/56-FAM/ | Not |
| qPCR probe | TCAAGGGCA/ZEN/TCCTGGGCTACA | applicable |
| | C/3IABkFQ/-3′ | |

The primers and probe used to quantify EF1A1 are shown in Table 2.

TABLE 2

| Target gene | | Amplicon |
|---|---|---|
| gDNA | Primer/qPCR Probe sequence | size |
| Human | F: 5′-CCACTGGAAGCAGGAATGAG | 100 bp |
| Ef1a1 | T-3′ | |
| primers | R: 5′-TGGTGCTCAAGCCACAGTT | |
| | G-3′ | |
| Human | 5′-/56-FAM/AC TTC CTG T/ | Not |
| Ef1a1 | ZEN/G AAA CCC AGT GTC | applicable |
| qPCR probe | TT/3IABkFQ-3′ | |

In some embodiments, other unique primers and probes may be used. qPCR reaction master mix is prepared by mixing qPCR master mix with forward primer, reverse primer and qPCR probe. For example, a qPCR reaction master mix is prepared by mixing qPCR master mix, forward primer, reverse primer and qPCR probe. In some embodiments, a total of about 0.01 μl-8 ml of qPCR reaction master mix is prepared by mixing about 0.01 μl-2 ml of qPCR master mix, 0.01 μl-2 ml of forward primer (0.01 μM—30 μM, preferably, 8 μM), 0.01 μl-2 ml of reverse primer (0.01 μM—30 μM, preferably, 8 μM) and 0.01 μl-2 ml of qPCR probe. In a particular embodiment, a total of about 11.8 μl of qPCR reaction master mix is prepared by mixing about 10 μl of qPCR master mix, 0.75 μl of forward primer (8 μM), 0.75 μl of reverse primer (8 μM) and 0.3 μl of qPCR probe (0.15 μM). The forward primer, the reverse primer and the qPCR probe correspond to the same target gene gDNA.

Subsequently, the final eluate or final supernatant from the Sample is added to the qPCR reaction master mix (Sample Reaction Mix). In some embodiments, 0.01 μl-5 ml of the final eluate from the Sample is added to the qPCR reaction master mix. In particular embodiment, 8.2 μl of the final eluate from the Sample is added to the qPCR reaction master mix.

A series of dilutions of gDNA purified from Reference are also prepared for the establishment of the Reference Standard Curve. The dilution is prepared by adding the final eluate or final supernatant of the Reference to 20 μl autoclaved Milli-Q water for 5-fold serial dilutions (5-1 to 5-5 serial dilutions). In some embodiments, the dilution is prepared by adding 0.01 μl-5 ml final eluate or final supernatant of the Reference to 0.04 μl-20 ml autoclaved Milli-Q water. In a particular embodiment, the dilution is prepared by adding 5 μl final eluate or final supernatant of the Reference to 20 μl autoclaved Milli-Q water. As a result, six dilutions (5⁰, 5¹, 5², 5³, 5⁴, 5⁵-fold dilutions) are set. Each of the dilutions is added into a separate qPCR reaction master mix (Reference Reaction Mix).

For example, if the number of cells used for purifying gDNA in the known sample is 5×10⁶ in the particular embodiment, the dilution series will be as follows:

TABLE 3

| Dilution | No. of cells of gDNA |
|---|---|
| 5⁰ | 5 × 10⁶ |
| 5⁻¹ | 1 × 10⁶ |
| 5⁻² | 2 × 10⁵ |
| 5⁻³ | 4 × 10⁴ |
| 5⁻⁴ | 8 × 10³ |
| 5⁻⁵ | 1.6 × 10³ |

In some embodiments, the serial dilution may be any number of fold. Also, the total volume of the qPCR reaction master mix may be varied.

Optionally, qPCR negative control may be prepared and undergoes qPCR with the Sample Reaction Mix and the Reference Reaction Mix. The qPCR negative control is prepared by adding Milli-Q water into the qPCR reaction master mix (Negative Control Reaction Mix).

qPCRs of Sample Reaction Mix, Reference Reaction Mixes and optionally Negative Control Reaction Mix are then concurrently carried out. In some embodiments, all of the Mixes are run in at least duplicates to minimize error. In some embodiments, the Mixes are loaded onto a multiwall plate to run the qPCR concurrently. In some embodiments, the qPCRs of Sample Reaction Mix, Reference Reaction Mix and optionally Negative Control Reaction Mix are carried out separately

The qPCRs are carried out under the following conditions.

Initial denaturation is performed for 1-3 cycles at 85° C.-100° C. for 1-10 minutes at a ramp rate at about 3-5° C./second, preferably, 1 cycle at 95° C. for about 3 minutes at a ramp rate at about 4.4° C./second.

qPCR is carried out for 30-50 cycles, preferably for 45 cycles, wherein each cycle include the steps of (i) heating the mixture to 85° C.-100° C. for 1-50 seconds at a ramp rate at about 3-5° C./second, preferably, 95° C. for about 15 seconds at a ramp rate at about 4.4° C./second, and (ii) incubating the mixture to 40° C.-80° C. for 1-120 seconds at a ramp rate at about 3-5° C./second, preferably, 55° C. for about 60 seconds at a ramp rate at about 2.2° C./second. Signals from the amount of qPCR product from the Sample, Reference and/or Negative Control are collected at the completion of all qPCR cycles. In some embodiments, the analysis mode of the qPCR system is set to quantification and the acquisition mode is set to single.

Cooling is carried out for 1-3 cycles which includes cooling it to 10° C.-1° C. for 1-10 minutes at a ramp rate at about 0.1-4° C./second, preferably, 1 cycle, which includes cooling it to 4° C. at a ramp rate at about 2.2° C./second.

Upon the completion of Probe-based qPCRs, determine the validity of the Probe-based qPCRs run by the following criteria: (i) efficiency being between 190-210%; (ii) the error of the standard curve is equal to or lower than 0.02.

The Reference Standard Curve gives correlations between the log concentration and the Crossing Point. The number of cells in the cell culture in a Sample (i.e. the cells with the 3D scaffold containing an unknown number of cells) may be determined by reference to Crossing Point of the Reference Standard Curve. The crossing point of the Reference Standard Curve may provide the estimated log concentration, thereby the number of cells in the Sample can be deduced. The number of cells/mg of the scaffold may be also deduced using the Reference Standard Curve.

From the present teachings, it may be seen that the method of the present invention does not rely on imaging techniques, cells embedded in opaque or translucent scaffold materials may be quantified. Furthermore, there is no size limit for the scaffold being studied; the entire scaffold may be processed for cell quantification. This improves data accuracy and reproducibility by accounting for all the cells embedded in the scaffold (even when cells are buried deep inside the scaffold) and by overcoming the problem of uneven cell distribution encountered during imaging. The proposed method greatly reduces the overall time and manpower required as there is no need for post-imaging analysis. Finally, this method is a high throughput assay; hundreds to thousands of samples may be simultaneously quantified.

The present invention may be used to quantify cell growth in cultivated meat or tissue transplants.

### EXAMPLES

Quantifying Cells Using Probe-Based qPCR Assay

The probe-based qPCR of the present invention is specific for amplifying a region of the human Gapdh genomic DNA (gDNA) sequence. In this example, HEK-293 cells were used. The HEK-293 cells were lysed and the genomic DNA (gDNA) thereof was obtained through Spin Column Extraction. Five-fold serial dilutions of gDNA samples were prepared, from 5×10⁶ cells-8×10³ cells (as shown in Table 3). The dilutions were loaded for Probe-based qPCR. Human Gapdh gDNA was amplified by the Probe-based qPCR assay.

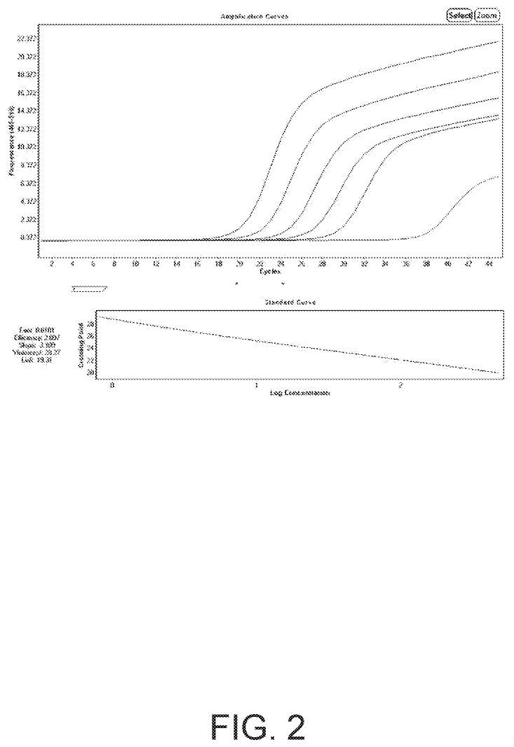

Referring to FIG. 2, amplification curves in a probe-based qPCR assay of five-fold serial dilutions of HEK-293 cells and a standard curve thereof are shown. The standard curve shows an amplification efficiency of 209.7% and an error of 0.0101, both of which are acceptable according to qPCR guidelines (efficiency=190%-210%; error 0.02). The Probe-based qPCR assay showed a linear dynamic range from 8×10³ cells-5×10⁶ cells. The standard curve also shows that there is a linear relationship between the log concentration and the Crossing Point.

Quantifying cells in sodium alginate scaffold using Probe-based qPCR assay

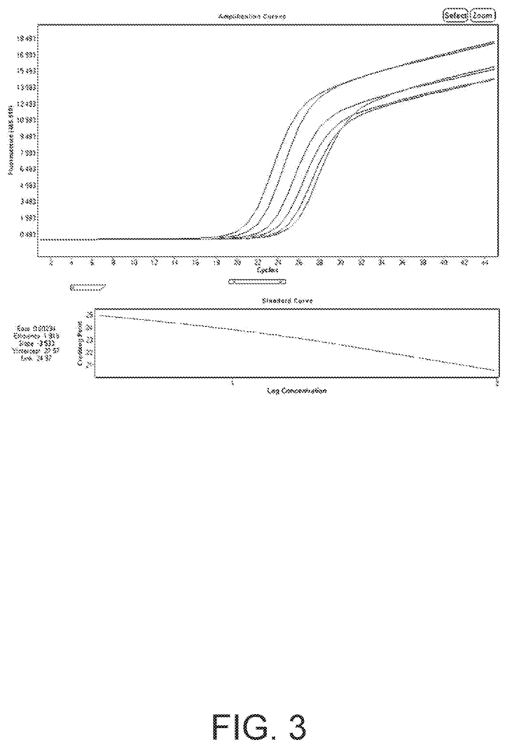

The Probe-based qPCR of the present invention may be used to quantify cells within 3D scaffolds. In this example, specified numbers of HEK293 cells (1×10⁶-3.125×10⁴) were embedded in sodium alginate scaffolds. Referring to FIG. 3, the gDNA from the cells was obtained through Spin

Columns Extraction. This extraction method reliably isolated gDNA from sodium alginate scaffolds. The gDNA samples were loaded for Probe-based qPCR. Amplification curves and the standard curve are obtained. The amplification efficiency was 191.9%; the error for the standard curve was <0.01. The standard curve also shows that there is a substantially linear relationship between the log concentration and the Crossing Point.

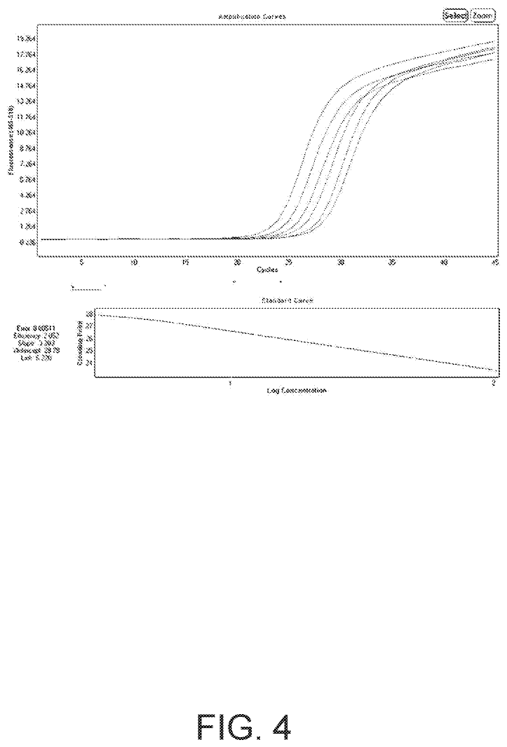

Referring to FIG. 4, the gDNA from the cells was obtained through Alkaline Extraction. This extraction method reliably isolated gDNA from sodium alginate scaffolds. The gDNA samples were loaded for Probe-based qPCR. Amplification curves and the standard curve are obtained. The amplification efficiency was 205.2%; the error for the standard curve was <0.01. The standard curve also shows that there is a substantially linear relationship between the log concentration and the Crossing Point.

Quantifying Cells in Denatured Collagen Scaffold Using Probe-Based qPCR Assay

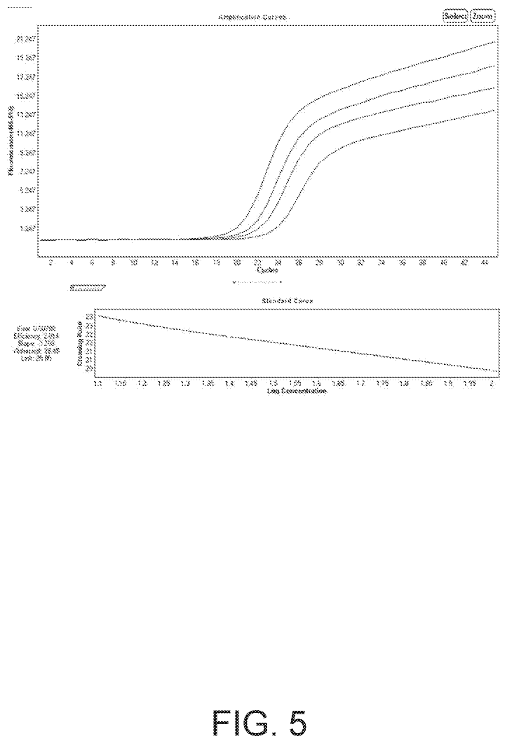

The Probe-based qPCR of the present invention may be used to quantify cells within 3D scaffolds. In this example, specified numbers of HEK293 cells (1×10⁶-1.25×10⁵) were embedded in denatured collagen scaffolds. Referring to FIG. 5, the gDNA from the cells was obtained through Spin

Columns Extraction. This extraction method reliably isolated gDNA from denatured collagen scaffolds. The gDNA samples were then loaded for Probe-based qPCR. Amplification curves and the standard curve are obtained. The amplification efficiency was 201.4%; the error in the standard curve was <0.01. The standard curve also shows that there is a substantially linear relationship between the log concentration and the Crossing Point.

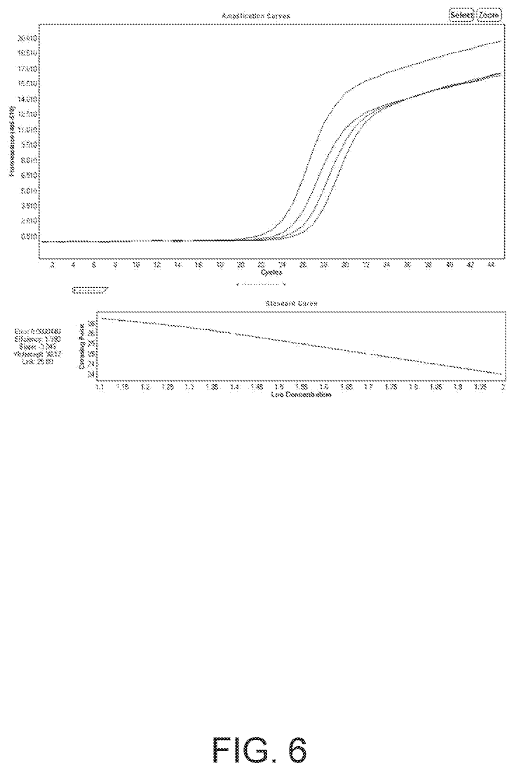

Referring to FIG. 6, the gDNA from the cells was obtained through Alkaline Extraction. This extraction method reliably isolated gDNA from denatured collagen scaffolds. The gDNA samples were then loaded for Probe-based qPCR. Amplification curves and the standard curve are obtained. The amplification efficiency was 199%; the error in the standard curve was <0.001. The standard curve also shows that there is a substantially linear relationship between the log concentration and the Crossing Point.

Comparison between Spin Column Extraction and Alkaline Extraction

Comparing with two extraction methods to isolate gDNA from the scaffolds for Probe-based qPCR, the Spin Column Extraction is ideal for purifying gDNA from complex scaffolds containing high levels of PCR inhibitors and impurities. However, it is more expensive than Alkaline Extraction and has upper limits in the number of cells for extraction (<500 ug DNA/column) and in the size of the scaffold (difficulty in diluting and transferring large volumes of sticky and viscous cell lysate). Alkaline Extraction is less costly than the Spin Column Extraction as all cells from the entire scaffold can be directly lysed in NaOH (no limit for cell number and size).

Monitoring Cell Growth within Denatured Collagen Scaffold Using Probe-Based qPCR Assay

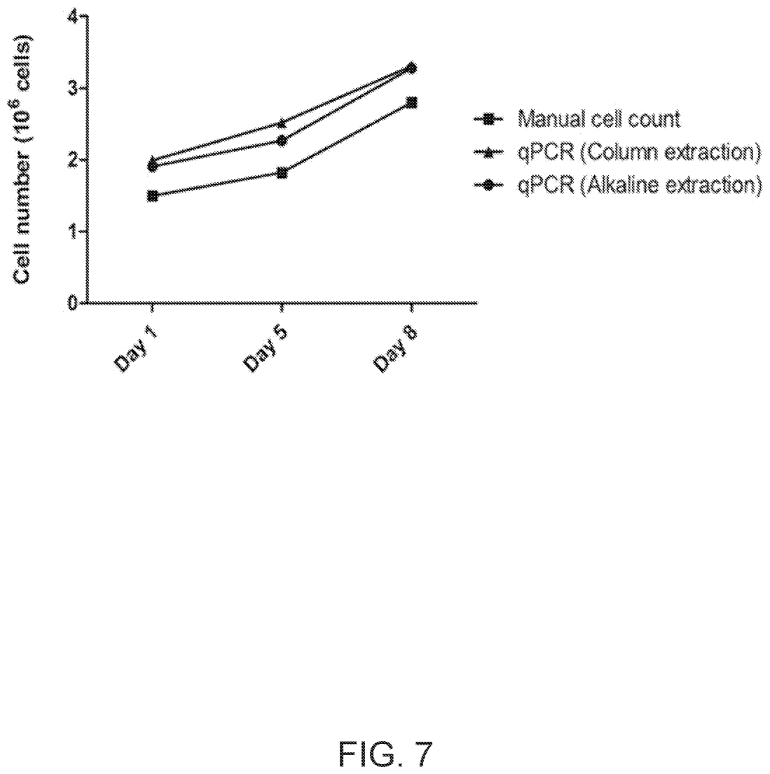

Referring FIG. 7, a Probe-based qPCR assay is used to measure cell growth within a denatured collagen scaffold. Cells were seeded onto individual scaffolds (2×10⁶ cells/scaffold, 1×1 cm scaffold) and samples were taken on days 1, 5, and 8 for manual cell count or gDNA extraction via Spin Column Extraction or Alkaline Extraction. Manual cell counting showed that the number of cells increased from 1.50×10⁶ cells on day 1 to 2.80×10⁶ cells on day 8, indicating that cell growth occurred in the denatured collagen scaffold. Similar trends in cell growth were also detected by Probe-based qPCR assay based on gDNA samples prepared by the Spin Column Extraction (day 1: 1.99×10⁶ cells; day 8: 3.31×10⁶ cells) and by Alkaline Extraction (day 1: 1.91×10⁶ cells; day 8: 3.28×10⁶ cells).

The discrepancies in the cell number between manual cell count and Probe-based qPCR approach might be because not all cells could be recovered from the scaffold by trypsinization for cell count. Moreover, some cells were damaged or fragmented during the trypsinization process; we only counted the viable whole cells. In contrast, the Probe-based qPCR approach accounts for all the cells because gDNA can be extracted from every cell embedded within the scaffold.

The above description is illustrative and is not restrictive. Many variations of embodiments may become apparent to those skilled in the art upon review of the disclosure. The scope embodiments should, therefore, be determined not with reference to the above description, but instead should be determined with reference to the pending claims along with their full scope or equivalents.

One or more features from any embodiment may be combined with one or more features of any other embodiment without departing from the scope embodiments. A recitation of “a”, “an” or “the” is intended to mean “one or more” unless specifically indicated to the contrary. Recitation of “and/or” is intended to represent the most inclusive sense of the term unless specifically indicated to the contrary.

While the present disclosure may be embodied in many different forms, the drawings and discussion are presented with the understanding that the present disclosure is an exemplification of the principles of one or more inventions and is not intended to limit anyone embodiment to the embodiments illustrated.

The disclosure, in its broader aspects, is therefore not limited to the specific details, representative system and methods, and illustrative examples shown and described above. Various modifications and variations may be made to the above specification without departing from the scope or spirit of the present disclosure, and it is intended that the present disclosure covers all such modifications and variations provided they come within the scope of the following claims and their equivalents.

### Exemplary Protocols

### A. Quantification of Cells

Cells embedded and cultured within a scaffold may display faster proliferation. This protocol aims to estimate the number of cells embedded in a scaffold at different time points.

Cell seeding into sodium alginate scaffold
1. Human embryonic kidney 293 (HEK293) cells were obtained from ATCC. Maintain HEK293 cells in complete medium (DMEM/F12, 10% FBS, 1% Glutamax, 0.2% Primocin) at 34° C. inside a 5% CO₂ incubator. When cells reach ˜80% cell confluence, subculture the cells at a split ratio of 1:10-1:20.
2. Prepare 2% sodium alginate (Sigma Aldrich) in PBS, 0.6 M CaCO₃ and 1.2 M glucono-O-lactone (GDL) in autoclaved MilliQ water. Sterilize 2% sodium alginate and GDL by passing them through a 0.2 μm filter. Autoclave 0.6 M CaCO₃ (note: CaCO₃ does not dissolve). Use these preparations on the same day of cell seeding.
3. When cells reach ˜80% cell confluence, detach cells by trypsinization and resuspend the cell pellet in a 1 ml complete medium. Determine the cell concentration using a hemocytometer.
4. To seed cells into sodium alginate, combine sodium alginate, cell suspension, CaCO₃, and GDL at a volume ratio of 10:8:1:1 (refer to steps 5-9 below as an example). The scaffold will gelatinize within 30 min to form a 1% sodium alginate gel which embeds the cells under physiological pH (˜pH 7).
5. Dilute the cell suspension to the desired cell concentration with complete medium (e.g. 1×10⁵-1.25×10⁷ cells/ml) and combine 80 ul of suspension (8 volumes) with 100 ul of sodium alginate (10 volumes). Mix by pipetting.
6. Since CaCO₃ does not completely dissolve, vortex CaCO₃ for 5 seconds and immediately add 10 ul (1 volume) to the mixture in step 5. Mix by pipetting.
7. Add 10 ul (1 volume) of GDL to the mix in step 6. Mix by pipetting.
8. Gelation begins immediately after the addition of GDL and completes within 30 minutes. Total scaffold volume will be ˜200 ul containing 8×10³-1×10⁶ cells.
9. To culture cells with the scaffold, add a complete medium and return the cells to the incubator. Alternatively, the scaffold can be directly processed for genomic DNA extraction.

Cell Seeding into Denatured Collagen Scaffold
1. Aseptically transfer the denatured collagen scaffold (Tantti Laboratory Inc) into Ultra-Low attachment multi-well plates or dishes.
2. When cells reach ˜80% cell confluence, detach cells by trypsinization and resuspend the cell pellet in a 1 ml complete medium. Determine the cell concentration using a hemocytometer.
3. Dilute the cell suspension to the desired cell seeding concentration (e.g. 2×10⁶ cells/cm 2 of scaffold) with a complete medium. For every 1 cm 2 of the scaffold, the maximum amount of cell suspension for cell seeding is 200 ul.
4. Slowly add 100 ul-200 ul of the diluted cell suspension to the center of the scaffold. Add half of the cell suspension and allow the scaffold the absorb all the contents before adding the other half of the cell suspension.
5. Return the cells to the incubator. To culture cells with the scaffold, add complete medium at 6-18 hours post-seeding. Alternatively, the scaffold can be processed for genomic DNA extraction at 6-18 hours post-seeding.
6. To recover cells from the scaffold for manual cell counting, use scissors and forceps to cut the scaffold into small pieces. Collect all the pieces into a 50 ml tube and trypsinize them for 10-15 minutes at 37° C. (vortex for <5 seconds every 5 minutes). When the scaffold has dissolved, centrifuge the tube at 400×g for 5 minutes and aspirate the supernatant. Resuspend the pellet in 1 ml complete medium and proceed to cell counting.

Genomic DNA Extraction—by Purelink Spin Column
1. Pre-weigh a clean 1.5 ml microcentrifuge tube. Transfer a small slice of scaffold (˜0.5 cm×0.5 cm×0.5 cm) or divide the entire scaffold to separate tubes. Weigh the tube(s) again. Calculate the weight of the scaffold.
2. Freeze and store the tubes at −20° C.
3. Thaw the tubes at room temperature. Mince the scaffold with a 1 ml pipette tip. Add 180 ul PureLink Genomic Digestion Buffer and 20 ul Proteinase K. Make sure the entire slice of the scaffold is submerged.
4. Heat the mixture at 55° C. for 1-16 hours, with occasional vortexing (<10 s). Ensure that the scaffold is dissolved after digestion.
5. Centrifuge the tube at 14000×g for 3 minutes. Transfer the supernatant to a clean 1.5 ml microcentrifuge tube.
6. Add 20 ul RNase A. Incubate at room temperature for 2 minutes.
7. Add 1 volume of PureLink Genomic Lysis/Binding Buffer (˜200 ul). Vortex for 5 seconds.
8. Add 1 volume of absolute ethanol (˜200 ul). Vortex for 5 seconds.
9. Transfer the mixture to a PureLink Spin Column. If precipitate has formed in step 8, centrifuge the tube at 14000×g for 5 minutes and transfer the supernatant to the PureLink spin column.
10. Centrifuge at 10000×g for 1 minute. Transfer the Spin Column to a new collection tube.
11. Add 500 ul Wash Buffer 1 with ethanol to the column. Centrifuge the column at 10000×g for 1 minute. Transfer the spin column to a new collection tube.
12. Add 500 ul Wash Buffer 2 with ethanol to the column. Centrifuge the column at 10000×g for 1 minute. Transfer the spin column to a new collection tube. Centrifuge the column at 10000×g for 3 minutes to remove residual wash buffer.
13. Transfer the spin column to a new collection tube. Elute DNA by adding 200 ul autoclaved MQ water to the column. Incubate at room temperature for 1 minute. Centrifuge the tube at 14000×g for 2 minutes. Save the eluate (Eluate 1).
14. Repeat step 13 twice to produce eluate 2 and eluate 3.
15. Combine all eluates. Over 95% of the purified genomic DNA will have been eluted from the column.

Genomic DNA Extraction—by Alkaline Extraction
1. Pre-weigh a clean 1.5 ml microcentrifuge tube. Transfer a small slice of scaffold (˜0.5 cm×0.5 cm×0.5 cm). Weigh the tube again. Calculate the weight of the scaffold. Alternatively, larger scaffolds (e.g., the entire scaffold) may be processed for alkaline extraction. Determine the weight of the scaffold if necessary.
2. Freeze and store the scaffold at −20° C.
3. Thaw the scaffold at room temperature. Mince the scaffold with a 1 ml pipette tip and/or scissors. Add 600 ul NaOH (50 mM) to the tube. Make sure the entire scaffold is submerged (increase the volume of NaOH for larger scaffolds).
4. Heat the mixture at 98° C. for 30 minutes, with occasional vortexing (<10 s).
5. Add 1 volume (e.g., 600 ul) autoclaved Milli Q H20. Vortex the tube (<10 s).
6. Add 0.1 volume (e.g., 60 ul) Tris-HCL (1 M, pH 8.0). Vortex the tube (<10 s).
7. Centrifuge the tube at 14000×g for 5 minutes. Transfer the supernatant to a clean tube.
8. (10×) Dilute the cellular DNA sample (e.g. 100 ul) with autoclaved MilliQ water (e.g. 900 ul) before loading it to qPCR plate.

Probe-Based Quantitative PCR (qPCR)
1. Quantify cellular genomic DNA using a probe-based quantitative real-time PCR assay in a LightCycler 480 System (Roche Diagnostics). Design primers (target amplicon size: 80-200 bp) and qPCR probes specific to the gene of interest (verify primer and probe specificities by blasting the sequences against the NCBI genome database). For human cells, use the primers and probes below.

TABLE 4

| Target gene | | Amplicon |
|---|---|---|
| gDNA | Primer/qPCR Probe sequence | size |
| Human | F: 5′-TCAAGAAGGTGGTGAAGCAG | 117 bp |
| Gapdh | G-3′ | |
| primers | R: 5′-CGTCAAAGGTGGAGGAGTG | |
| | G-3′ | |
| Human | 5′-/56-FAM/ | Not |
| Gapdh | TCAAGGGCA/ZEN/TCCTGGGCTACA | applicable |
| qPCR | C/3IABkFQ/-3′ | |
| probe | | |

Note: The double-quenched human Gapdh qPCR probe contains a 5′ fluorophore (FAM), an internal ZEN quencher, and a 3′ Iowa Black™ FQ (IBFQ) quencher.
2. Set up the qPCR reaction as follows:

TABLE 5

| | Volume per | |
|---|---|---|
| | reaction | |
| PrimerTime Gene Expression Master Mix | 10 | ul |
| (Integrated DNA Technologies) | | |
| Forward primer (8 uM) | 0.75 | ul |
| Reverse primer (8 uM) | 0.75 | ul |
| qPCR probe (150 nM) | 0.3 | ul |
| Total | 11.8 | ul |

Since each sample and standard should be run in duplicates, the total number of reactions required should be (no. samples+no. of standards+PCR negative control)×2 replicates+2 spare reactions (for pipetting error). Prepare a master mix and keep it on ice.
3. Perform 5-fold serial dilutions (5-1 to 5-5 serial dilutions) of the reference gDNA standard by adding 5 ul gDNA to 20 ul autoclaved MQ water. The reference gDNA should be extracted by the same method as the unknown samples. There will be six standards (5⁰, 5¹, 5², 5³, 5⁴, 5⁵-fold dilutions) for establishing the standard curve in the qPCR run.
   For example, if the number of cells used for purifying gDNA in the Reference Sample is 5×10⁶, the dilution series will be as follows:

TABLE 6

| Dilution | No. of cells of gDNA |
|---|---|
| 5⁰ | 5 × 10⁶ |
| 5⁻¹ | 1 × 10⁶ |
| 5⁻² | 2 × 10⁵ |
| 5⁻³ | 4 × 10⁴ |
| 5⁻⁴ | 8 × 10³ |
| 5⁻⁵ | 1.6 × 10³ |

4. Write down what is added to each well of the qPCR plate on an experiment record sheet.
5. Add 11.8 ul of PCR reaction master mix per well of a LightCycler Multiwell plate 96 (Roche). Avoid introducing bubbles.
6. Add 8.2 ul of an unknown sample, standard, or MQ water (PCR negative control) into each well according to the experiment record sheet. Avoid introducing bubbles.
7. Cover the qPCR plate with a sealing foil supplied in the LightCycler Multiwell plate 96 pack (Roche). Make sure all the wells are tightly sealed by the sealing foil to prevent evaporation during qPCR.
8. Collect the well contents to the bottom of the wells by centrifuging at 200×g inside a Beckman Coulter Allegra 25R Centrifuge (Beckman Coulter Life Sciences).
9. Place the qPCR plate into the Roche LightCycler 480. Set the PCR conditions as follows:
   Set the excitation and emission filters: FAM dye
      Initial Denaturation:
      95° C. 3 minutes (Ramp Rate 4.4° C./sec)
      1 cycle
      PCR
      Analysis Mode: Quantification
      95° C. 15 seconds (Ramp Rate 4.4° C./sec)
      55° C. 1 minutes (Ramp Rate 2.2° C./sec, Acquisition Mode: Single)
      45 cycles
      Cooling
      37° C. ∞(Ramp Rate 2.2° C./sec)
      1 cycle
10. Begin the qPCR run.
11. After the qPCR run is complete, take the qPCR plate out. Verify the validity of the qPCR run by the following criteria:
   The efficiency is 190-210%,
   The error of the standard curve is 0.02
12. Deduce the number of cells from the standard curve. Calculate the number of cells/mg of the scaffold. Alternatively, if the scaffold changes volume or density upon prolong culture, or if cells may not be evenly distributed throughout the scaffold, it may be more accurate to extract gDNA from the entire scaffold and determine the total number of cells.

The following replaces all previous versions of the claims:

1. A method for quantifying cells embedded in a scaffold, comprising:
(a) a lysing step to cause the cells to lyse, wherein the lysing step comprises the step of heating the cells together with the scaffold in the presence of a proteinase with a buffer or alkali;
(b) a purification step comprising performing at least three times of elution and collecting all the eluates or performing centrifugation, thereby capturing at least 90% of genomic DNA released from the cells; and
(c) a quantification step comprising the steps of (i) forming a sample reaction mix by adding a predetermined volume of the collected eluate or supernatant into a qPCR master mix; (ii) forming a plurality of reference reaction mixes wherein each of the reference reaction mixes contains genomic DNA purified from a known number of cells diluted to different concentration; (iii) carrying out probe-based qPCR on the sample reaction mix and the plurality of reference reaction mixes; (iv) collecting the signals from the probe-based qPCR products from the sample reaction mix and reference reaction mixes; (v) creating a standard curve based on the collected signals from the reference reaction mix wherein the standard curve provides a correlation between the number of cells and the collected signals; and (vi) deducing the number of cells in the sample through the standard curve.

2. The method of claim 1, wherein the quantification step further comprises forming a negative control reaction mix by adding Milli-Q water into the probe-based qPCR reaction master mix.

3. The method of claim 2, wherein the lysing step comprises the step of heating the cells together with the scaffold in the presence of alkali; and the purification step comprising centrifugation and collecting supernatant, wherein the alkali is sodium hydroxide.

4. The method of claim 2, wherein the lysing step comprises the step of heating the cells together with the scaffold in the presence of a proteinase with a buffer; and the performing at least three times of elution.

5. (canceled)

6. The method of claim 5, wherein the probe-based qPCR is carried out to quantify GAPHD genomic DNA.

7. The method of claim 5, wherein the probe-based qPCR is carried out to quantify EF1A genomic DNA.

8. The method of claim 3, wherein the quantification step further comprises carrying out probe-based qPCR in a multiwell plate.

9. The method of claim 4, wherein the quantification step further comprises carrying out probe-based qPCR in a multiwell plate.


SEQUENCE LISTING (6 sequences)

SEQ ID NO: 1 - DNA, 21 bp, Human
tcaagaaggtggtgaagcagg

SEQ ID NO: 2 - DNA, 20 bp, Human
cgtcaaaggtggaggagtgg

SEQ ID NO: 3 - DNA, 21 bp, Human
ccactggaagcaggaatgagt

SEQ ID NO: 4 - DNA, 20 bp, Human
tggtgctcaagccacagttg

SEQ ID NO: 5 - DNA, 21 bp, Human
tcaagaaggtggtgaagcagg

SEQ ID NO: 6 - DNA, 20 bp, Human
cgtcaaaggtggaggagtgg